(19) United States
(12) Patent Application Publication
(10) Pub. No.: US 2006/0193825 A1
(43) Pub. Date: Aug. 31, 2006

(54) Pharmaceutical formulations for sustained drug delivery

(75) Inventors: Gary F. Musso, Hopkinton, MA (US); Nicholas Barker, Southborough, MA (US); Janet L. Wolfe, Newton, MA (US); Ming Ye, Acton, MA (US)

Correspondence Address: LAHIVE & COCKFIELD, 28 STATE STREET, BOSTON, MA, 02109, US

(73) Assignee: Praecis Phamaceuticals, Inc., Waltham, MA (US)

(21) Appl. No.: 11/265,553

(22) Filed: Nov. 2, 2005

Related U.S. Application Data
(63) Continuation-in-part of application No. 11/205,292, filed on Aug. 15, 2005.
Application No. 11/205,292 is a continuation-in-part of application No. 10/835,717, filed on Apr. 29, 2004.
(60) Provisional application No. 60/466,388, filed on Apr. 29, 2003.

(51) Int. Cl. A61K 38/28; A61K 38/22; A61K 38/36; A61K 38/37; A61K 38/19; A61K 38/20; A61K 38/21; A61K 31/717; A61K 38/16; A61K 38/00

(52) U.S. Cl. 424/85.1; 424/85.2; 424/85.7; 514/3; 514/8; 514/12; 514/54; 514/57

(57) ABSTRACT

The present invention provides pharmaceutical formulations comprising a solid ionic complex of a polypeptide having an isoelectric point lower than physiological pH and an anionic carrier molecule. The formulations of the invention are suitable as depot formulations for the sustained release of therapeutic polypeptides.

### RELATED APPLICATIONS

This application is a continuation-in-part application of U.S. patent application Ser. No. 11/205,292, filed on Aug. 15, 2005, pending, which is a continuation-in-part application of U.S. patent application Ser. No. 10/835,717, filed on Apr. 29, 2004, pending, which claims priority to U.S. Provisional Patent Application Ser. No. 60/466,388, filed on Apr. 29, 2003, the entire contents of which are incorporated herein by reference.

### BACKGROUND

A variety of diseases and clinical disorders are treated by the administration of a pharmaceutically active peptide.

In many instances, the therapeutic effectiveness of a pharmaceutically active peptide depends upon its continued presence in vivo over prolonged time periods. To achieve continuous delivery of the peptide in vivo, a sustained release or sustained delivery formulation is desirable, to avoid the need for repeated administrations. One approach for sustained drug delivery is by microencapsulation, in which the active ingredient is enclosed within a polymeric membrane to produce microparticles. Additional sustained delivery formulations for administering pharmaceutically active peptides in vivo continuously for prolonged time periods are needed.

### SUMMARY

The present invention provides pharmaceutical formulations comprising a solid ionic complex of a polypeptide having an isoelectric point lower than physiological pH and an anionic carrier molecule. The formulations of the invention are suitable as depot formulations for the sustained release of therapeutic polypeptides.

### DETAILED DESCRIPTION OF THE INVENTION

The present invention provides pharmaceutical compositions comprising a solid ionic complex of a polypeptide having an isoelectric point lower than physiological pH and an anionic carrier molecule suitable for the sustained release of a pharmaceutically active compound in vivo. The invention further provides methods of making and using the sustained release pharmaceutical compositions of the invention. The advantages of the pharmaceutical compositions of the invention include the ability for delivery of a pharmaceutically active compound, either systemically or locally, for prolonged periods (e.g., several weeks, one month or several months) and the ability to load high concentrations of the pharmaceutically active compound into the solid ionic complex that is formed.

The polypeptide can be, for example, a monomeric or multimeric protein having a therapeutic activity. Preferred polypeptides can have a molecular weight of 100,000 daltons or less, 50,000 daltons or less, 40,000 daltons or less, 30,000 daltons or less, 20,000 daltons or less, 10,000 daltons or less, 5,000 daltons or less or 2,000 daltons or less. For example, the polypeptide can be composed of 2 or more, preferably five or more, amino acid residues. In one embodiment, the polypeptide comprises a single peptide chain composed of 1000 or fewer amino acid residues. In another embodiment, the polypeptide comprises a peptide chain composed of from about 5 to about 50 amino acid residues. The polypeptide can also comprise two or more peptide chains which are joined together covalently, for example, by disulfide bridges. Each of these chains can be composed of from about 5 to about 1000 amino acid residues, from about 5 to about 500 residues, from about 5 to about 300 residues or from about 5 to about 100 residues. Particular polypeptides which can be formulated as described herein include, but are not limited to, peptide hormones, enzymes useful for enzyme replacement therapy, non-naturally occurring peptides and protein fragments having useful therapeutic activity, cytokines, lymphokines and chemokines having isoelectric points below physiological pH.

Polypeptides which can be formulated according to the present invention include polypeptides having an isoelectric point which is below physiological pH. As used herein, the term “physiological pH’ refers to a pH of 7.4. Preferably, the polypeptide has an isoelectric point less than about 7.0, less than about 6.5 or less than about 6.0. In preferred embodiments, the polypeptide has an isoelectric point which is between about 4.0 and about 7.0, more preferably between about 4.5 and about 6.5, and most preferably between about 5.0 and about 6.5. For example, the polypeptide can have a pI of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8 or 6.9.

A variety of pharmaceutically active peptides may be used in the formulations. Non-limiting examples of such peptides include peptides that contain one or more lysine and/or arginine residues and lysine-like and/or arginine-like amino acid residues, such as LHRH analogues, recombinant luteinizing hormone, e.g., lutropin alpha, bradykinin analogues, parathyroid hormone, adenocorticotrophic hormone (ACTH), calcitonin, vasopressin analogues (e.g., 1-deamino-8-D-arginine vasopressin (DDAVP)), and synthetic forms of vasopressin, e.g., Desmopressin Acetate. Other non-limiting examples of pharmaceutically active peptides that can be used in the formulations and methods of the invention include octreotide, endorphin, liprecin, erythropoietin, protamine, platelet aggregation inhibitor (epoprostenol), platelet glycoprotein IIb/IIIa receptor, recombinant platelet glycoprotein IIb/IIIa receptor antibodies, e.g., Abciximab and Eptifibatide, angiotensin II, antidiuretic hormone, neurotrophic factors, keratinocyte growth factor, leukemia inhibiting factor, monocyte chemoattractant protein-1, endothelial growth factors, thymosin alpha 1, thymosin alpha 1 IIb/IIa inhibitor, thymosin beta 10, thymosin beta 9, thymosin beta 4, alpha-1 antitrypsin, phosphodiesterase (PDE) compounds, VLA-4 (very late antigen-4), VLA-4 inhibitors, bisphosponates, respiratory syncytial virus antibody, e.g., antibodies directed against the epitope in the A antigenic site of the F protein of respiratory syncytial virus (RSV), e.g., PALIVIZUMAB, cystic fibrosis transmembrane regulator (CFTR) protein, deoxyreibonuclease (Dnase), bactericidal/permeability increasing protein (BPI), anti-CMV antibody, oxytocin, growth hormones, e.g., somatotropin, pituitary hormones, somatostatin, asparaginase, chorionic gonadotropin, growth hormone releasing hormone, growth hormone releasing peptide, interferons (e.g., interferons α, β, γ, interferon β-1a, interferon α-2a, interferon alfacon-1, interferon alpha-n3 (Human Leukocyte Derived), colony stimulating factor, bone morphogenic proteins (BMP) (e.g., 1, 2, 3, 4, 5, 6, and 7), interleukins (e.g., interleukin-1, -2, -3, -4, -5, -6, -7, -8, -9, -10, -11, -13, -14, -15, -16, -17, -18, -19, -20, -21, -22, -23, -24, -25, -26, -27, -28, -29 and -30), e.g., recombinant interleukin antibodies, e.g., IL-2, e.g., Aldesleukin, e.g., recombinant interleukins, e.g., IL-11, e.g., oprelvekin, e.g., interleukin receptor antagonists, e.g., IL-1 receptor antagonist, e.g., anakinra, glucocerebrosidase, e.g., Imiglucerase, macrophage activating factor, macrophage peptide, B cell factor, T cell factor, protein A, suppressive factor of allergy, cell necrosis glycoprotein, immunotoxin, lymphotoxin, tumor necrosis factor, tumor inhibitory factor, transforming growth factor, HER2, e.g., antibodies against HER2, e.g., Trastuzumab, myelin, e.g., synthetic forms or fragments thereof, e.g., Glatiramer Acetate, alpha-1 antitrypsin, albumin, apolipoprotein-E, apolipoprotein A1, erythropoietin, hyper-glycosylated erythropoietin, factor VII, factor VIII, factor IX, plasminogen activator, urokinase, streptokinase, protein C, activated Protein C, e.g., Drotrecogin alpha, protein S, C-reactive protein, renin inhibitor, collagenase inhibitor, superoxide dismutase, leptin, platelet derived growth factor, epidermal growth factor, epidermal growth factor receptor (EGFR), e.g., recombinant EGFR antibodies, e.g., Cetuximab, osteogenic growth factor, osteogenesis stimulating protein, calcitonin, insulin, insulin analogs, e.g., Insulin Glulisine and Insulin Glargine, amylin, e.g., synthetic analogues thereof, e.g., Pramlintide, atriopeptin, cartilage inducing factor, connective tissue activator protein, follicle stimulating hormone, luteinizing hormone, FSH releasing hormone, nerve growth factor, parathyroid hormone, or a portion thereof, e.g., Teriparatide, prostoglandin, relaxin, secretin, somatomedin, insulin-like growth factor, thrombolytics, pamiteplase, lanoteplase, and teneteplase; nerve growth factor (NGF), osteoprotegerin, Rhdnase, e.g., domase alpha and Tenecteplase, erythropoiesis stimulating protein (NESP), coagulation factors such as Factor V, Factor VII, Factor VIIa, Factor VIII, Factor IX, Factor X, Factor XII, Factor XIII, von Willebrand factor; ceredase, cerezyme, alpha-glucosidase, collagen, cyclosporin, alpha defensins, beta defensins, exedin-4, thrombopoietin (TPO), heparin, human serum albumin, low molecular weight heparin (LMWH), alpha-1 proteinase inhibitor, elcatonin, fibrinogen, filgrastim (granulocyte colony-stimulating factor, e.g., Sargramostim), adrenocorticotrophic hormone, glucagon, glucagon-like peptide 1 (GLP-1) receptor or agonists thereof, e.g., Exendin-4, glucagon-like peptide 1, or analogues thereof, e.g., Exenatide, cholecystokinin, pancreatic polypeptide, gastrin releasing peptide, corticotropin releasing factor, or analogues thereof, e.g., Corticorelin Ovine Triflutate, thyroid stimulating hormone, TNF receptor (e.g., TNFR(P75) and TNFR(P55)), IL-1 receptor antagonist (e.g., IL1-Ra), cell surface antigen (e.g., CD2, 3, 4, 5, 7, 11a, 11b, 18, 19, 20, 23, 25, 33, 38, 40, 45 and 69), e.g., recombinant CD20 antibodies, e.g., Rituximab, TNF-α, e.g., recombinant TNFα antibodies, e.g., Infliximab, Etanercept, NF-κB, urate oxidase, e.g., Rasburicase, cone snail peptide w-cenotoxin M-VII-A, e.g., Ziconotide, antimicrobial antifungal and antibacterial analogues, non-limiting examples of which include, Caspofingin acetate, ADENOREGULIN, Aureins, Gaegurins, Thanatin, Ranatuerin-2CB, Ranatuerin-2CA, Cecropin A, Cecropin B, Melittin B, Indolicidin, Tritipticin, Androctonin, Tachystatin A, Dermaseptins, Gomesin, Hepcidin 20, Hepcidin 25, Peptide PGQ, Protegrins, RatNPs Seminalplasmin, Tracheal antimicrobial peptide, Dolabellanin B2, AFP1, AFP2, Dermaseptin BI, Buforin I, Buforin II, Histones, Opistoporins, Ponericins, Penaeidins, Spingerin, Skin peptide tyrosine-tyrosine, Lingual antimicrobial peptide, Tricholongin, Termicin, Holotricins, Penaeidins, Nk-Lysin, Magainin 2, Neutrophil defensins, Cyclic Defensin, Alpha-basrubrin, Melanotropin alpha (Alpha-MSH), Brevinin, Pseudins (1, 2, 3, 4), Anti-fungal protein 1(pafp-s), Misgurin, P-18, Pseudo-hevein (Minor hevein), MUC7 20-Mer, Histatins (3, 5, 8), Nigrocin, lactoferrin (Lf), Ranalexin, antiviral analogues, e.g., Antiviral protein Y3, Alloferon 1, Lactoferricin B, hexapeptide, Tricyclic peptide RP, Indolicidin, GNCP-2, GNCP-1, HNP-1 Defensin, HNP-2 Defensin, Defensin, CORTICOSTATIN III (MCP-1), CORTICOSTATIN IV (MCP-2), NP-3A defensin, Protegrin 2, Protegrin 3, Protegrin 3, Protegrin 4, Protegrin 5, RatNP-1, RatNP-2, RatNP-3, RatNP-4, Caerin 1.1, Circulin A (CIRA), Circulin B (CIRB), Cyclopsychotride A (CPT), Ginkbilobin, Alpha-basrubrin, Enfuvirtide, or other antiretroviral agents. Fragments, analogues, derivatives, e.g., peptidomimetics, of any of the foregoing peptidic compounds may be used in the pharmaceutical formulations of the present invention. Monoclonal antibodies, polyclonal antibodies, antibody fragments, and virus-derived vaccine antigens raised against any of the foregoing peptidic compounds are also contemplated for use in the pharmaceutical formulations of the present invention.

Suitable peptides further include sequence variants and other analogues of the specific polypeptides set forth above having desirable therapeutic activity. For example, variants having structural modifications which result in an improved property, such as increase stability, bioavailability or therapeutic activity, or decreased side effect profile, are included. Such variants include sequence variants, in which one or more amino acid residues of the parent polypeptide have been replaced with another amino acid residue, such as a conservative substitution or a non-natural amino acid residue. The variant can also be a fragment of the parent polypeptide, resulting, for example, from the removal of one or more amino acid residues at the N- and/or C-terminus of the parent polypeptide.

Polypeptides which can be formulated as described herein further include synthetic polypeptides which include one or more non-naturally occurring amino acid residues, such as L-amino acid residues having non-natural side chains or D-amino acid residues. Suitable polypeptides can further include polypeptides which comprise one or more peptidomimetic units, for example, one or more dipeptide, tripeptide, or tetrapeptide mimetic units as known in the art.

Further, the invention provides, in at least one embodiment, a pharmaceutical formulation comprising a solid ionic complex of a polypeptide having an isoelectric point higher than the physiological pH and in ionic carrier molecule. In a specific exemplification of this embodiment, the polypeptide can be somatostatin, or a synthetic polypeptide analogue of somatostatin, e.g., octreotide.

Polypeptides which are suitable for use in the present invention can be identified using methods known in the art. The isoelectric point of a polypeptide can be determined experimentally, for example, via isoelectric focusing, in which a polypeptide migrates in a pH gradient under the influence of an applied electric field. At its isoelectric pH (“isoelectric point” or “pI”) the polypeptide has no net electric charge and stops moving. The isoelectric point of a polypeptide can also be estimated theoretically based on the amino acid sequence of the polypeptide. Such calculated isoelectric points, however, fail to account for post-translational modifications, such as glycosylation, and the effects of the local environment on the pKa of amino acid side chains, which can significantly alter the acidity of a functional group.

The anionic carrier macromolecule is preferably a linear or cross-linked polymer comprising monomers which bear a negative charge at physiological pH. In one embodiment, each of the monomeric units in the polymer comprises an acidic functional group or a salt thereof. In another embodiment, a fraction of the monomers within the polymer are functionalized with an acidic functional group. Preferably, the polymer comprises either anionic functional groups or cationic functional groups, although the polymer can comprise both cationic and anionic functional groups, so long as the proportion of these groups allows for the desired net anionic charge at physiological pH. Each of the cationic or anionic groups in the polymer can be the same or different, although in preferred embodiments they are the same.

In one embodiment, the polymer includes acidic or anionic functional groups, such as carboxylate, sulfonate, phosphonate, sulfate ester, phosphate ester, sulfamate or carbamate groups. Preferably the anionic groups are carboxylate groups.

The anionic carrier macromolecule is physiologically compatible and is, preferably, biodegradable or bioresorbable.

As used herein, the term “administering to a subject” is intended to refer to dispensing, delivering or applying a composition (e.g., pharmaceutical formulation) to a subject by any suitable route for delivery of the composition to the desired location in the subject, including delivery by either the parenteral or oral route, intramuscular injection, subcutaneous/intradermal injection, intravenous injection, buccal administration, transdermal delivery, administration by the rectal, colonic, vaginal, intranasal, respiratory tract, intrathecal, or intracerebral route, administration to cells in ex vivo treatment protocols, topical delivery, and delivery on a surface, e.g., a biocompatible surface, for example on the surface of a surgically implanted device, e.g., a stent, shunt, or catheter. Preferred anionic carrier macromolecules are suitable for administration via intraperitoneal, intramuscular or intravenous injection or inhalation. Suitable anionic polymers include anionic polysaccharides; anionic polyesters; anionic polyamides, for example, anionic peptides; and polyacrylates.

Examples of suitable anionic polymers include, but are not limited to, carboxymethylcellulose, poly(glutamic acid), poly(aspartic acid), poly(glutamic acid-co-glycine), poly(aspartic acid-co-glycine), poly(glutamic acid-co-alanine), poly(aspartic acid-co-alanine), starch glycolate, polygalacturonic acid, poly(acrylic acid), and alginic acid.

In another embodiment, the carrier macromolecule may be dextran sulfate, croscarmellose sodium, carbomers (poly(acrylic acid)), sodium hyaluronate, xanthan gum, or chitosan.

Preferred anionic polymers include anionic polysaccharides and anionic polypeptides. The anionic polymer can be linear or cross-linked. For example, the anionic polymer can be cross-linked to varying extents, for example, via ionic cross-linking or covalent cross-linking. In one embodiment, the anionic polymer bears a net anionic charge and is cross-linked by the addition of an amount of a cationic cross-linking polymer. The relative amounts of the two polymers can be varied to provide different degrees of cross-linking, but should be such that the combination retains a net ionic charge sufficient to bind a desired amount of the polypeptide. For example, an anionic polymer, such as carboxymethylcellulose, can be cross-linked with varying amounts of a cationic polymer, such as poly(lysine).

In another embodiment, the anionic polymer is covalently cross-linked. In a first example, an anionic polymer comprising carboxylate groups is covalently cross-linked as is known in the art by reacting a fraction of the carbosylate groups, or activated derivatives thereof, with a suitable cross-linking reagent such as a dialcohol, an aminoalcohol or a diamine, under conditions suitable for forming ester and/or amide linkages. In this case, the ionic polymer will comprise carboxylate groups and ester/amide groups, with the ester/amide groups on one polymer strand linked to ester/amide groups on another polymer strand by bridging groups derived from the dialcohol, amino alcohol or diamine used. Preferably, the dialcohol, amino alcohol or diamine is pharmaceutically acceptable.

The solid ionic complex an have a range of compositions. For example, the complex can comprise from about 2% polypeptide to about 95% polypeptide. The complex can comprise from about 98% anionic macromolecule to about 5% anionic macromolecule. Preferably, the solid ionic complex comprises 10% or greater, 20% or greater or 30% or greater polypeptide. More preferably, the solid ionic complex comprises 40% or greater or 50% or greater polypeptide. Preferably, the solid ionic complex comprises 90% or less; 80% or less; or 70% or less anionic macromolecule. More preferably, the solid ionic complex comprises 60% or less or 50% or less anionic macromolecule. All percentages disclosed herein are weight/weight unless otherwise indicated.

The ratio (weight/weight) of the polypeptide to the ionic macromolecule in the solid ionic complex of the invention is preferably about 10, 9, 8, 7, 6, 5, 4, 3, 2, 1, 0.75, 0.5, 0.25, or 0.1. Preferably the ratio of the polypeptide to the ionic macromolecule is about 0.5, 0.75, 1 or greater.

In one embodiment, the solid ionic complex consists essentially of the anionic macromolecule and the polypeptide. Typically, such a solid ionic complex will be hydrated and the mass of the complex will include some amount of water. The degree of hydration can be determined by subjecting the complex to dehydrating conditions, preferably conditions under which the polypeptide and the anionic macromolecule are stable, and determining the resulting weight decrease.

In another embodiment, the solid ionic complex comprises a polypeptide having an isoelectric point below physiological pH, the anionic carrier macromolecule and one or more additional substances. Suitable additional substances include a second pharmaceutically active compound, which, preferably, has a net positive change at physiological pH. The additional substance or substances can also include one or more pharmaceutically acceptable excipients or other agents which modulate the properties of the complex, such as solubility.

The solid ionic complex is, preferably, substantially insoluble in aqueous solvent at physiological pH. The term “substantially insoluble” is used herein to refer to a material that has limited solubility under a given set of conditions. It is to be understood that a substantially insoluble material can have finite solubility, but generally is soluble to an extent providing a concentration of pharmaceutically active agent no greater than 10 mM, 1 mM, 100 μM, 10 μM or 1 μM. For a given polypeptide, the anionic carrier macromolecule and additional excipients, if any, can be selected to optimize the properties of the solid ionic complex with respect to aqueous solubility and/or polypeptide content, among others. For example, cross-linking is expected to reduce the solubility of the resulting complexes and can be accomplished using methods known in the art, such as covalent cross-linking or ionic cross-linking, as discussed above.

The solubility of the solid ionic complex can also be modulated by including in the complex an excipient such as one or more di- or trivalent metal cations, such as Al³⁺, Ca²⁺, Fe²⁺, Fe³⁺ or Mg²⁺. The metal cation can be added in varying amounts as required to obtain the desired solubility. For example, the metal cation(s) can be added in an amount required to neutralize from 0.01% to 50% of the anionic groups on the anionic carrier macromolecule. Preferably, the metal cation is added in an amount required to neutralize from 0.01% up to 2%, 5%, 7%, 10%, 12%, 15%, 17% or 20% of the anionic groups on the anionic carrier macromolecule. One of skill in the art can readily determine a combination of excipients, cross-linking agents and extent of cross-linking to provide a complex having the desired solubility.

The present invention further includes pharmaceutical compositions comprising a solid ionic complex of a pharmaceutically active compound and an ionic carrier molecule and a pharmaceutically acceptable carrier. For example, the solid ionic complex can be suspended in a vehicle suitable for injection, water for injection, a buffered aqueous solution, or an oil-based vehicle.

In addition to the water-insoluble complex, the pharmaceutical formulations of the invention can comprise additional pharmaceutically acceptable carriers and/or excipients. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for topical, oral, buccal, vaginal, rectal, pulmonary, nasal, transdermal, intravenous, intramuscular, subcutaneous, intrathecal, intracerebral, or parenteral administration (e.g., by injection). Excipients include pharmaceutically acceptable stabilizers and disintegrants. The use of such media and agents for pharmaceutically active substances is well known in the art. Except insofar as any conventional media or agent is incompatible with the peptidic compound, use thereof in the pharmaceutical formulations is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral, nasal, transdernal (topical), transmucosal, rectal, transvaginal, or buccal administration.

Pharmaceutical formulations suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the formulation must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifingal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the formulation. Solutions or suspensions for parenteral, intradermal, or subcutaneous administration may also include antioxidants such as ascorbic acid or sodium bisulfite, chelating agents such as ethylenediaminetetraacetic acid, buffers such as acetates, citrates or phosphates, and agents for the adjustment of tonicity such as sodium chloride or dextrose. pH can be adjusted with acids or bases, such as hydrochloric acid or sodium hydroxide. The parenteral formulation can be enclosed in ampoules, disposable syringes or multiple dose vials made of glass or plastic.

Sterile injectable solutions can be prepared by incorporating the water-insoluble complex in the required amount in an appropriate solvent with one or a combination of ingredients enumerated above, as required, followed by an appropriate sterilization method, such as, for example, filter sterilization, gamma-irradiation, and the like. In one embodiment, dispersions are prepared by incorporating the water-insoluble complex of the invention into a sterile vehicle which contains a basic dispersion medium and the required other ingredients from those enumerated above. In the case of sterile powders for the preparation of sterile injectable solutions, the methods of preparation may be vacuum drying and freeze-drying which yields a powder of the the water-insoluble complex of the invention plus any additional desired ingredient from a previously sterile-filtered solution thereof. Other compositions useful for attaining systemic delivery of the water-insoluble complex of the invention include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

The compounds of the invention may also be formulated as depot preparations. Such formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example as a sparingly soluble salt.

Peroral pharmaceutical formulations of the water-insoluble complex of the invention include liquid solutions, emulsions, suspensions, and the like. The pharmaceutically acceptable carriers suitable for preparation of such formulations are well known in the art. Typical components of carriers for syrups, elixirs, emulsions and suspensions include ethanol, glycerol, propylene glycol, polyethylene glycol, liquid sucrose, sorbitol. and water. For a suspension, typical suspending agents include methyl cellulose, sodium carboxymethyl cellulose, tragacanth, and sodium alginate; typical wetting agents include lecithin and polysorbate 80; and typical preservatives include methyl paraben and sodium benzoate. Peroral liquid formulations may also contain one or more components such as sweeteners, flavoring agents and colorants.

Oral formulations generally include an inert diluent or an edible carrier. They can be enclosed in capsules (e.g., gelatine, cellulosic, or pullulan capsules), or compressed into tablets. For the purpose of oral administration, the water-insoluble complex of the invention can be incorporated with excipients and used in the form of tablets, troches, or capsules. Oral formulations can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the formulation. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In solid dosage forms for oral administration (capsules, tablets, pills, dragees, powders, granules and the like), the water-insoluble complex is mixed with one or more pharmaceutically-acceptable carriers. In the case of capsules, tablets and pills, the pharmaceutical formulations may also comprise buffering agents. Solid formulations of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the water-insoluble complex thereof moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Systemic administration of the water-insoluble complex of the invention can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal, e.g., intranasal, administration can be accomplished through the use of, for example, nasal sprays, nasal drops, or powders.

Transmucosal formulations for rectal or vaginal administration may be presented as a suppository or retention enema, which may be prepared by mixing the water-insoluble complex of the invention with one or more suitable non-irritating excipients or carriers comprising, for example, cocoa butter, polyethylene glycol, a suppository wax or a salicylate. Such excipients or carriers are generally solid at room temperature, but liquid at body temperature, and therefore, they will melt in the rectum or vaginal cavity and release water-insoluble complex.

The transdermal formulations of this invention can also be administered topically to a subject via percutaneous passage of the formulation into the systemic circulation of the subject., e.g., by the direct laying on or spreading of the formulation on the epidermal or epithelial tissue of the subject. Topical administration can also involve the use of transdermal administration such as transdermal patches or iontophoresis devices. Such compositions include, for example, lotions, creams, solutions, gels and solids. These topical compositions may comprise an effective amount, usually at least about 0.1%, or evan from about 1% to about 5%, of a water-insoluble complex of the invention. Suitable carriers for topical administration typically remain in place on the skin as a continuous film, and resist being removed by perspiration or immersion in water. Generally, the carrier is organic in nature and capable of having dispersed or dissolved therein the water-insoluble complex. The carrier may include pharmaceutically acceptable emolients, emulsifiers, thickening agents, solvents and the like. Other components can be incorporated into the transdermal patches as well. For example, formulations and/or transdermal patches can be formulated with one or more preservatives or bacteriostatic agents including, but not limited to, methyl hydroxybenzoate, propyl hydroxybenzoate, chlorocresol, benzalkonium chloride, and the like.

Dosage forms for topical administration of the water-insoluble complex can include creams, pastes, sprays, lotions, gels, ointments, eye drops, nose drops, ear drops, suppositories, and the like. In such dosage forms, the water-insoluble complex of the invention can be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the formulations can contain polyethylene glycol 400. They can be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, can be impregnated with the compositions in solution, lotion, cream, ointment or other such form can also be used for topical application.

Pharmaceutical formulations are also provided which are suitable for administration as an aerosol, by inhalation. For administration by inhalation, the water-insoluble complex may be delivered in the form of an aerosol spray from pressured container or dispenser which contains a suitable propellant, e.g., a gas such as carbon dioxide, or a nebulizer.

Dry Powder formulations for inhalation may be delivered using any suitable dry powder inhaler (DPI), i.e., an inhaler device that utilizes a subject's inhaled breath as a vehicle to transport the dry powder pharmaceutical formulation to the lungs. Examples of such devices are Inhale Therapeutic Systems' dry powder inhalation devices as described in Patton, J. S., et al., U.S. Pat. No. 5,458,135, Oct. 17, 1995; Smith, A. E., et al., U.S. Pat. No. 5,740,794, Apr. 21, 1998; and in Smith, A. E., et. al., U.S. Pat. No. 5,785,049, Jul. 28, 1998, herein incorporated by reference. When administered using a device of this type, the powdered formulation is contained in a receptacle having a puncturable lid or other access surface, preferably a blister package or cartridge, where the receptacle may contain a single dosage unit or multiple dosage units. Convenient methods for filling large numbers of cavities (i.e., unit dose packages) with metered doses of dry powder formulation are described, e.g., in Parks, D. J., et al., International Patent Publication WO 97/41031, Nov. 6, 1997, incorporated herein by reference.

Other dry powder dispersion devices for pulmonary administration of dry powders include those described, for example, in Newell, R. E., et al, European Patent; No. EP 129985, Sep. 7, 1988); in Hodson, P. D., et al., European Patent No. EP472598, Jul. 3, 1996; in Cocozza, S., et al., European Patent No. EP 467172, Apr. 6, 1994, and in Lloyd, L. J. et al., U.S. Pat. No. 5,522,385, Jun. 4, 1996, incorporated herein by reference. Also suitable for delivering the dry powders of the present invention are inhalation devices such as the Astra-Draco “TURBUHALER”. This type of device is described in detail in Virtanen, R., U.S. Pat. No. 4,668,218, May 26, 1987; in Wetterlin, K., et al., U.S. Pat. No. 4,667,668, May 26, 1987; and in Wetterlin, K., et al., U.S. Pat. No. 4,805,811, Feb. 21, 1989, all of which are incorporated herein by reference. Other suitable devices include dry powder inhalers such as Rotahaler((Glaxo), DiscustD (Glaxo), Spiros_inhaler (Dura Pharmaceuticals), and the Spinhaler (Fisons). Also suitable are devices which; employ the use of a piston to provide air for either entraining powdered formulation , lifting formulation from a carrier screen by passing air through the screen, or mixingair with powder formulation in a mixing chamber with subsequent introduction of the powder to the subject through the mouthpiece of the device, such as described in Mulhauser, P., et al, U.S. Pat. No. 5,388,572, Sep. 30, 1997, incorporated herein by reference.

The water-insoluble complex of the present invention may also be delivered using a pressurized, metered dose inhaler (MDI), e.g., the Ventolin metered dose inhaler, or a nebulizer, containing a solution or suspension of water-insoluble complex in a pharmaceutically inert liquid propellant, e.g. dichlorodifluoromethane, trichlorofluoromethane, dichlorotetrafluoroethane, tetrafluoroethane, heptafluoropropane, carbon dioxide or other suitable gas., as described in Laube, et al., U.S. Pat. No. 5,320,094, Jun. 14, 1994, and in Rubsamen, R. M., et al, U.S. Pat. No. 5,672,581 (1994), both incorporated herein by reference. Nebulizers for delivering an aerosolized solution include the AERx_(Aradigm), the Ultravent (Mallinkrodt), the Pari LC Plus_or the Pari LC Star_(Part GmbH, Germany), the DeVilbiss Pulmo-Aide, and the Acorn II (Marquest Medical Products). In the case of a pressurized aerosol the dosage unit may be determined by providing a valve to deliver a metered amount. Capsules and cartridges of, e.g., gelatin for use in an inhaler or insulator may be formulated containing a powder mix of a water-insoluble complex of the invention and a suitable powder base such as lactose or starch.

According to yet another embodiment, the water-insoluble complex of this invention may be incorporated into compositions for coating an implantable medical device, such as prostheses, artificial valves, vascular grafts, stents and catheters (such as, balloon catheters and indwelling catheters), and/or shunts, including mechanical shunts. Suitable coatings and the general preparation of coated implantable devices are described in U.S. Pat. Nos. 6,099,562; 5,886,026; and 5,304,121, the disclosures of which are incorporated herein by reference. The coatings typically comprise biocompatible polymeric materials such as a hydrogel polymer, polymethyldisiloxane, polycaprolactone, polyethylene glycol, polylactic acid, ethylene vinyl acetate, and mixtures thereof. The implantable medical devices useful in the methods of the present invention can be metallic or plastic, and may comprise a biodegradable coating or porous non-biodegradable coating.

In one embodiment, the water-insoluble complex of the invention is coated on a medical device, e.g., a stent, implanted into a subject during a medical procedure, such as, for example, angioplasty. In one embodiment, the pharmaceutically active compound incorporated into the water-soluble complex and coated on the medical device implanted into a subject prevents restenosis following the placement of the medical device in the subject. In one embodiment, restenosis is inhibited by inhibiting late-stage endothelialization

In another embodiment, the water-insoluble complex of the invention is irreversibly bonded to a medical device, e.g., a stent, implanted into a subject during a medical procedure, such as, for example, angioplasty. Without wishing to be bound by theory, the irreversible bonding of the water-insoluble complex to the medical device may not only reduce restenosis, but may also encourage encapsulation of the carrier macromolecule and the stent into the vessel wall such that the carrier macromolecule is unavailable for release into the bloodsteam and potentially form emboli or accumulate in the liver or spleen as circulating particulate matter. Accordingly, in one embodiment, restenosis is enhanced by promoting early stage re-endothelialization.

Non-limiting examples of pharmaceutically active peptidic compounds that are suitable for incoporation into a water-insoluble complex and coated or irreversibly bound on a medical device and implanted in a subject during a medical procedure, include angiogenesis inhibitors, such as Angiostatin, Endostatin, Interleukin 12, Recombinant human platelet factor 4(rPF4), Thrombospondin, and TNP-470; vascular smooth muscle cell anti-proliferative agents, such as transforming growth factor beta; anti-thrombogenic agents such as urokinase, and PPACK (dextrophenylalanine proline arginine chloromethylketone); angiogenic and anti-angiogenic agents; agents blocking smooth muscle cell proliferation such as angiopeptin and monoclonal antibodies capable of blocking smooth muscle cell proliferation; antineoplastic/antiproliferative/anti-mitotic agents such as endostatin and angiostatin; anesthetic agents such as L-arginine; anti-coagulants such as D-Phe-Pro-Arg chloromethyl ketone, an RGD peptide-containing compound, platelet receptor antagonists, anti-thrombin antibodies, thrombin inhibitors, e.g., Bivalirudin, anti-platelet receptor antibodies, vascular cell growth promotors such as growth factors, growth factor receptor antagonists; vascular cell growth inhibitors such as growth factor inhibitors, growth factor receptor antagonists, inhibitory antibodies, antibodies directed against growth factors, bifunctional molecules consisting of a growth factor and a cytotoxin, bifunctional molecules consisting of an antibody and a cytotoxin; survival proteins which protect against cell death, such as anti-apoptotic Bcl-2 family factors and Akt kinase; bone morphogenic proteins (BMP) (e.g., 1, 2, 3, 4, 5, 6, and 7); and combinations thereof.

The pharmaceutical formullation of the invention may also be administered intrathecally into the cerebrospinal fluid (CSF). The intrathecal administration of the water-insoluble complex of the present invention may comprise introducing the pharmaceutical formulaltion into a cerebral ventricle. Alternatively, the intrathecal administration may comprise introducing the pharmaceutical formulaltion into the lumbar area. In yet another alternative, the intrathecal administration comprises introducing the pharmaceutical composition into the cisterna magna. Any such administration is-preferably via a bolus injection. In other embodiments, the intrathecal administration is achieved by use of an infusion pump.

The administration of the pharmaceutical formulations of the invention may also be intracerebrally. Administration may be by, for example, direct intracerebral administration, or by, for example, stereotactic microinjection.

Intracerebral administration, may be provided by perfusion via a mechanized delivery system, such as an osmotic pump, or by implantation.

Preparation of Compositions

The present invention also relates to a method of preparing a solid ionic complex comprising an ionic macromolecule and a pharmaceutically active compound. The solid ionic complex of the invention is prepared by combining the pharmaceutically active compound and the carrier macromolecule under conditions such that a water-insoluble complex of the pharmaceutically active compound and the ionic carrier macromolecule forms. In one embodiment, the method comprises the steps of (1) providing a polypeptide having an isoelectric point below physiological pH and an anionic carrier macromolecule; and (2) combining the polypeptide and the anionic carrier macromolecule under conditions such that a water-insoluble complex of the pharmaceutically active compound and the carrier macromolecule forms. Preferably, the polypeptide and the anionic macromolecule are combined in an aqueous solvent at a pH below the isoelectric point of the polypeptide. For example, the polypeptide and the anionic carrier macromolecule can be combined in solution in an aqueous buffer at a pH below the isoelectric point of the polypeptide. In one embodiment, the pH is no more than 2 pH units below the isoelectric point of the polypeptide; preferably the pH is no more than one pH unit below the isoelectric point of the polypeptide.

The anionic macromolecule can be combined with the polypeptide in a variety of ways. For example, a solution of the anionic macromolecule can be mixed with a solution of the polypeptide under conditions suitable for precipitation of the solid ionic complex. The two solutions can include the same solvent or different solvents. Preferably, if the solvents are different, they are miscible. Alternately, the anionic macromolecule can be added as a solid to a solution of the polypeptide or the polypeptide can be added to a solution of the ionic macromolecule.

In another embodiment, the ionic macromolecule and the polypeptide are added to a solvent in which neither is substantially soluble, but in which a by-product of the complexation, or ion-exchange process, is soluble. For example, a polypeptide which forms a water-insoluble hydrochloride salt can be added to an aqueous suspension of the sodium salt of an anionic macromolecule. The resulting suspension can be agitated for a sufficient period of time for formation of the desired solid ionic complex. In this case, the ion exchange process resulting in the desired solid ionic complex is driven, at least in part, by the solubility of the sodium chloride product.

Once the solid ionic complex precipitates, the precipitate can be removed from the solution by means known in the art, such as filtration (e.g., through a 0.45 micron nylon membrane), centrifugation and the like. The recovered paste than can be dried (e.g., in vacuo or in a 70° C. oven or a vacuum oven), and the solid can be milled or pulverized to a powder by means known in the art (e.g., hammer or gore milling, or grinding in mortar and pestle). Following milling or pulverizing, the powder can be sieved through a screen (preferably a 90 micron screen) to obtain a uniform distribution of particles. Moreover, the recovered paste can be frozen and lyophilized to dryness. The powder form of the complex can be dispersed in a carrier solution to form a liquid suspension or semi-solid dispersion suitable for injection. Accordingly, in various embodiments, a pharmaceutical formulation of the invention is a dry solid, a liquid suspension or a semi-solid dispersion. Examples of liquid carriers suitable for use in liquid suspensions include saline solutions, glycerin solutions, lecithin solutions and oils suitable for injection.

In another embodiment, the pharmaceutical formulation of the invention is a sterile formulation. For example, following formation of the water-insoluble complex, the complex can be sterilized, optimally by gamma irradiation or electron beam sterilization. Accordingly, the method of the invention for preparing a pharmaceutical formulation described above can further comprise sterilizing the water-insoluble complex by gamma irradiation or electron beam irradiation. Preferably, the formulation is sterilized by gamma irradiation using a gamma irradiation dose of at least 15 Kgy. In other embodiments, the formulation is sterilized by gamma irradiation using a gamma irradiation dose of at least 19 KGy or at least 24 Kgy. Alternatively, to prepare a sterile pharmaceutical formulation, the water-insoluble complex can be isolated using conventional sterile techniques (e.g., using sterile starting materials and carrying out the production process aseptically). Accordingly, in another embodiment of the method for preparing a pharmaceutical formulation described above, the water-insoluble complex is formed using aseptic procedures.

Use of Compositions

In another embodiment, the present invention relates to a method of administering a polypeptide to a subject, where the polypeptide has an isoelectric point which is lower than physiological pH. The method comprises the steps of (1) providing a pharmaceutical composition comprising a solid ionic complex comprising the polypeptide and an anionic carrier macromolecule and (2) contacting the body of the subject with the pharmaceutical composition. The body of the subject can be contacted with the pharmaceutical composition by a variety of methods. For example, the pharmaceutical composition can be injected into the subject's body. The injection can be, for example, an intramuscular, intravenous, intraperitoneal or subcutaneous injection. The subject can also be caused to inhale or swallow the pharmaceutical composition. The subject's eye or eyes can also be contacted with the pharmaceutical composition.

The invention further relates to a method of treating a subject suffering from a medical condition for which a polypeptide having an isoelectric point below physiological pH is indicated. The method comprises the steps of (1) providing a pharmaceutical composition comprising a solid ionic complex of the polypeptide and an anionic carrier macromolecule; and (2) administering the pharmaceutical composition to the subject.

The subject can be an animal in need of treatment for which the pharmaceutically active compound is indicated, and is preferably a mammal, such as a canine, feline, bovine, equine, ovine or porcine animal or a primate, such as a monkey, an ape or a human. More preferably, the subject is a human. The subject can be an individual diagnosed with, or suspected of having, the medical condition, or an individual at risk of developing the medical condition.

The term “medical condition”, as used herein, is a disease or disorder which is susceptible to medical treatment. The subject is in need of treatment for a medical condition if modification or prevention of the condition is desirable, or if the subject would benefit from alleviation of the symptoms of the condition. As intended herein, a polypeptide is “indicated” for a medical condition if it provides therapeutic benefit to an individual having the medical condition or is of use in prevention (prophylaxis) of the medical condition.

Devices which can be used to administer the pharmaceutical compositions of the invention are also contemplated. One suitable example of such a device is a syringe which houses a pharmaceutical composition comprising a solid ionic complex comprising the polypeptide and an anionic carrier macromolecule, where the complex is suspended in a vehicle suitable for injection. Another suitable example is an inhalation device which houses a pharmaceutical composition comprising a solid ionic complex comprising the polypeptide and an anionic carrier macromolecule and a pharmaceutically acceptable carrier suitable for inhalation. The inhalation device can be, for example, a dry powder inhaler, a nebulizer or a metered dose inhaler.

### EXEMPLIFICATION

Insulin Depot Formation and Characterization

Materials

Bovine insulin was obtained from Sigma Chemical Company (catalog no. I8405). Carboxymethylcellulose sodium (Low Viscosity, USP) was obtained from Spectrum Laboratory Products (Catalog no. CA 193; degree of substitution 0.84)

Preparation of Bovine Insulin-Carboxymethylcellulose Complex

Bovine insulin (756 mg) was dissolved in a minimal amount of 50% acetic acid in water and sufficient 5% acetic acid in water was added to obtain an insulin concentration of approximately 10 mg/mL. Sufficient 1% sodium hydroxide solution was then added to bring the pH to 3.9, resulting in an insulin concentration of 5.8 mg/mL. A 0.5% (weight/weight) solution of carboxymethylcellulose in water was prepared and filtered. 16 mL of the 0.5% CMC solution was added to the insulin solution with stirring and a white precipitate appeared immediately. After stirring for an additional hour, the precipitate was isolated by filtration and washed with water. An additional 100 mL water was added to the supernatant, causing the formation of more precipitate, which was isolated by centrifugation and washed with water. The wet solids were combined and dried in vacuo to yield 779 mg of a freely flowing white powder.

Analysis of the powder revealed the following composition (weight/weight): insulin 86.64%, CMC 7.50%; water 1.50%.

The solubility of the powder in a variety of media was determined and is shown in the table below

| Solvent | Solubility (mg complex/ML) |
|---|---|
| Water | 0.016 (0.014) |
| Ethanol | 0.012 (0.010) |
| 5% Dextrose | 0.018 (0.016) |
| Saline | 0.052 (0.045) |
| 0.33 M NaCl | 0.329 (0.285) |
| 5% Acetic Acid | 7.138 (6.182) |
| Organic Solvent | ≦0.01 mg/mL |
| (excluding DMSO) | |

1. A solid ionic complex comprising an anionic carrier macromolecule and a polypeptide having an isoelectric point less than about 7.4.

2. The solid ionic complex of claim 1 wherein the polypeptide has an isoelectric point less than about 7.0.

3. The solid ionic complex of claim 2 wherein the polypeptide has an isoelectric point less than about 6.5.

4. The solid ionic complex of claim 3 wherein the polypeptide has an isoelectric point less than about 6.0

5. The solid ionic complex of claim 4 wherein the polypeptide has an isoelectric point less than about 5.5.

6. The solid ionic complex of claim 5 wherein the polypeptide has an isoelectric point less than about 5.0.

7. The solid ionic complex of claim 1 wherein the polypeptide has an isoelectric point between about 4.5 and about 7.0.

8. The solid ionic complex of claim 7 wherein the polypeptide has an isoelectric point between about 5.0 and about 6.5.

9. The solid ionic complex of claim 1 wherein the anionic carrier macromolecule is a polypeptide or a polysaccharide.

10. The solid ionic complex of claim 9 wherein the anionic carrier macromolecule is selected from the group consisting of carboxymethylcellulose, poly(glutamic acid), poly(aspartic acid), poly(glutamic acid-co-glycine), poly(aspartic acid-co-glycine), poly(glutamic acid-co-alanine), poly(aspartic acid-co-alanine), starch glycolate, polygalacturonic acid, poly(acrylic acid) and alginic acid.

11. The solid ionic complex of claim 10 wherein the anionic carrier macromolecule is selected from the group consisting of poly(glutamic acid) and poly(aspartic acid).

12. The solid ionic complex of claim 10 wherein the anionic carrier macromolecule is carboxymethylcellulose.

13. The solid ionic complex of claim 1, wherein the polypeptide is selected from the group consisting of peptide hormones, enzymes useful for enzyme replacement therapy, non-naturally occurring peptides and protein fragments having useful therapeutic activity, cytokines, lymphokines and chemokines having isoelectric points below physiological pH.

14. The solid ionic complex of claim 1 wherein the polypeptide is selected from the group consisting of parathyroid hormone, adenocorticotrophic hormone (ACTH), calcitonin, 1-deamino-8-D-arginine vasopressin (DDAVP)), Desmopressin Acetate, octreotide, endorphin, liprecin, erythropoietin protamine, platelet aggregation inhibitor (epoprostenol), platelet glycoprotein IIb/IIIa receptor, recombinant platelet glycoprotein IIb/IIIa receptor antibodies, angiotensin II, antidiuretic hormone, neurotrophic factor, keratinocyte growth factor, leukemia inhibiting factor, monocyte chemoattractant protein-1, endothelial growth factor, thymosin alpha 1, thymosin alpha 1 IIb/IIa inhibitor, thymosin beta 10, thymosin beta 9, thymosin beta 4, alpha-1 antitrypsin, phosphodiesterase (PDE). VLA-4 (very late antigen-4), respiratory syncytial virus antibody, cystic fibrosis transmembrane regulator (CFTR) protein, deoxyreibonuclease (Dnase), bactericidal/permeability increasing protein (BPI), anti-CMV antibody, oxytocin, growth hormone, pituitary hormone, somatostatin, asparaginase, chorionic gonadotropin, growth hormone releasing hormone, growth hormone releasing peptide, interferons α, βγ, interferon β-1a, interferon α-2a, interferon alfacon-1, interferon alpha-n3, colony stimulating factor, bone morphogenic protein, interleukin, Aldesleukin, oprelvekin, anakinra, glucocerebrosidase, macrophage activating factor, macrophage peptide, B cell factor, T cell factor, protein A, suppressive factor of allergy, cell necrosis glycoprotein, immunotoxin, lymphotoxin, tumor necrosis factor, tumor inhibitory factor, transforming growth factor, HER2. Trastuzumab, myelin, Glatiramer Acetate, alpha-1 antitrypsin, albumin, apolipoprotein-E, apolipoprotein Al, erythropoietin, hyper-glycosylated erythropoietin, factor VII, factor VIII, factor IX, plasminogen activator, urokinase, streptokinase, protein C, activated Protein C. Drotrecogin alpha, protein S, C-reactive protein, renin inhibitor, collagenase inhibitor, superoxide dismutase, leptin, platelet derived growth factor, epidermal growth factor, epidermal growth factor receptor (EGFR), Cetuximab, osteogenic growth factor, osteogenesis stimulating protein, calcitonin, insulin, Insulin Glulisine and Insulin Glargine, amylin, Pramlintide, atriopeptin, cartilage inducing factor, connective tissue activator protein, follicle stimulating hormone, luteinizing hormone, FSH releasing hormone, nerve growth factor, parathyroid hormone, Teriparatide, prostoglandin, relaxin, secretin, somatomedin, insulin-like growth factor, thrombolytics, pamiteplase, lanoteplase, and teneteplase, nerve growth factor (NGF), osteoprotegerin, Rhdnase, dornase alpha, Tenecteplase, erythropoiesis stimulating protein (NESP), Factor V, Factor VIIa, Factor Factor X, Factor XII, Factor XIII, von Willebrand factor, ceredase, cerezyme, alpha-glucosidase, collagen, cyclosporin, alpha defensins, beta defensins, exedin-4, thrombopoietin (TPO), heparin, human serum albumin, low molecular weight heparin (LMWH), alpha-1 proteinase inhibitor, elcatonin, fibrinogen, filgrastim (granulocyte colony-stimulating factor), Sargramostim, adrenocorticotrophic hormone, glucagon, glucagon-like peptide 1 (GLP-1), Exendin-4, glucagon-like peptide 1, Exenatide, cholecystokinin, pancreatic polypeptide, gastrin releasing peptide, corticotropin releasing factor, Corticorelin Ovine Triflutate, thyroid stimulating hormone, TNF receptor. IL-1 receptor antagonist, cell surface antigen, Rituximab, TNF-α, Infliximab, Etanercept, NF-κB, urate oxidase, Rasburicase, cone snail peptide w-cenotoxin M-VII-A, Ziconotide, Caspofungin acetate, ADENOREGULIN, Aurein, Gaegurin, Thanatin, Ranatuerin-2CB, Ranatuerin-2CA. Cecropin A, Cecropin B. Melittin B, Indolicidin, Tritrpticin, Androctonin, Tachystatin A, Dermaseptins, Gomesin, Hepcidin 20, Hepcidin 25, Peptide PGQ. Protegrins, RatNPs Seminalplasmin, Tracheal antimicrobial peptide. Dolabellanin B2, AFP1, AFP2, Dermaseptin BI, Buforin I, Buforin II, Histone, Opistoporin, Ponericin, Penaeidin, Spingerin, Skin peptide tyrosine-tyrosine, Lingual antimicrobial peptide, Tricholongin, Termicin, Holotricins, Penaeidins. Nk-Lysin, Magainin 2, Neutrophil defensins, Cyclic Defensin, Alpha-basrubrin, Melanotropin alpha (Alpha-MSH). Brevinin, Pseudin, Anti-fungal protein 1(pafp-s), Misgurin, P-18, Pseudo-hevein (Minor hevein), MUC7 20-Mer, Histatins (3, 5, 8), Nigrocin, lactoferrin (Lf), Ranalexin, Antiviral protein Y3, Alloferon 1, Lactoferricin B, hexapeptide, Tricyclic peptide RP, Indolicidin, GNCP-2, GNCP-1, HNP-1 Defensin, HNP-2 Defensin, Defensin, CORTICOSTATIN III (MCP-1) CORTICOSTATIN IV (MCP-2), NP-3A defensin, Protegrin 2, Protegrin 3, Protegrin 3, Protegrin 4, Protegrin 5, RatNP-1, RatNP-2, RatNP-3, RatNP-4, Caerin 1.1, Circulin A (CIRA), Circulin B (CIRB), Cyclopsychotride A (CPT), Ginkbilobin, Alpha-basrubrin, and Enfuvirtide.

15. A pharmaceutical composition comprising the solid ionic complex of claim 1 and a pharmaceutically acceptable carrier.

16. The pharmaceutical composition of claim 15 wherein the pharmaceutically acceptable carrier is a liquid suitable for injection.

17. A method of administering a polypeptide to a subject, said polypeptide having an isoelectric point below physiological pH, comprising the steps of:
(a) providing a pharmaceutical composition comprising a solid ionic complex of claim 1; and
(b) contacting the subject's body with the pharmaceutical composition by a method selected from the group consisting of
(i) injecting the pharmaceutical composition into the subject's body;
(ii) causing the subject to inhale the pharmaceutical composition
(iii) causing the subject to swallow the pharmaceutical composition; and
(iv) contacting the eyes of the subject with the pharmaceutical composition.

18. A method of treating a subject having a medical condition for which a polypeptide having an isoelectric point below physiological pH is indicated, said method comprising the step of administering to the subject a therapeutically effective amount of a pharmaceutical composition comprising a solid ionic complex of claim 1.
